(19) United States
(12) Patent Application Publication
(10) Pub. No.: US 2007/0292305 A1
(43) Pub. Date: Dec. 20, 2007

(54) STERILIZATION OF MEDICAL DEVICES

(76) Inventors: Sinead Dempsey, Galway (IE); Jim Phelan, Galway (IE)

Correspondence Address: WORKMAN NYDEGGER, 1000 EAGLE GATE TOWER,, 60 EAST SOUTH TEMPLE, SALT LAKE CITY, UT, 84111, US

(21) Appl. No.: 11/758,917

(22) Filed: Jun. 6, 2007

Related U.S. Application Data
(60) Provisional application No. 60/804,366, filed on Jun. 9, 2006.

(51) Int. Cl. A61L 2/20

(52) U.S. Cl. 422/28; 422/33; 422/34

(57) ABSTRACT

The invention concerns methods for effectively sterilizing packaged or unpackaged medical devices such as drug- and/or polymer-coated stents. The method is well-suited to sterilizing medical devices that would be degraded by steam or irradiation sterilization. The present invention involves the placing of a packaged or unpackaged medical device into a pressure, temperature, and humidity controlled environment and exposing the device to a liquid or gaseous sterilizer for a given period of time. An example of a liquid or gaseous sterilizer is ethylene oxide. Given the right temperature, humidity, pressure, sterilizer concentration, and sufficient time, the sterilizer is able to effectively sterilize the device by contacting the exposed surfaces. In the case of packaged medical devices, the sterilizer contacts the device by diffusing through the packaging material.

### CROSS-REFERENCE TO RELATED APPLICATIONS

This application claims the benefit of U.S. Provisional Patent Application Ser. No. 60/804,366, filed Jun. 9, 2006, and entitled “STERILIZATION OF MEDICAL DEVICES,” with Sinead Dempsey and Jim Phelan as inventors, which is incorporated herein by specific reference.

### BACKGROUND OF THE INVENTION

I. The Field of the Invention

The present invention relates to methods for sterilizing packaged and unpackaged medical devices. More particularly, the present invention relates to improved methods for sterilization of packaged and unpackaged medical devices that are, for one reason or another, incompatible with heat, radiation, or other sterilization techniques.

II. The Related Technology

Many medical devices, such as endoscopes, percutaneous translumenal coronary angioplasty (“PTCA”) catheters, drug-coated and/or polymer-coated stents, stent delivery catheters, balloon catheters, and the like, must be sterilized prior to use. These medical devices must be thoroughly sterilized because they are, for example, inserted into body cavities and/or into the circulatory system where infection, as a result of using a contaminated medical device, could have serious consequences. Many of these medical devices are, however, incompatible with traditional methods of sterilization involving intense heat, exposure to high doses of ionizing radiation, or other harsh conditions.

Traditionally, many medical instruments have been sterilized through a combination of heat and pressure in an autoclave. An autoclave is a heavy-walled chamber capable of withstanding high heat and high pressure. Medical devices to be sterilized are placed in the chamber, the door is sealed, and the chamber is heated while also injecting large quantities of steam. Sterilization is achieved in an autoclave through a combination of high pressure, extreme heat, and moisture. While autoclaving is an effective sterilization technique, the environment in an autoclave is very harsh. As such, autoclaving may be inappropriate for some medical devices and/or medical device packaging. In addition, the high temperature that is produced during autoclave sterilization may cause some materials included in the medical devices and/or packaging to degrade or melt.

A typical alternative to autoclaving involves exposing objects to high doses of ionizing radiation in a process known as irradiation. In irradiation, objects are usually sterilized either by exposure to gamma radiation or by exposure to a high-energy electron beam. However, many medical devices are incompatible with irradiation because of the damage that can be caused by irradiation. For example, plastic medical devices, drug coatings, and/or polymer coatings subjected to irradiation sterilization may be unfavorably affected by the radiation and the environment used during sterilization, and may experience changes in the polymer structure such as chain scission or cross-linking.

Therefore, it would be advantageous to have a method of sterilizing medical devices that does not use excessively high heat or irradiation. Additionally, it would be advantageous to have a method of sterilizing medical devices that uses sterilizing gasses such as ethylene oxide (EtO).

### SUMMARY OF THE INVENTION

The present invention includes methods for sterilizing medical devices and/or medical device packaging. Such methods utilize a chemical sterilizer that sterilizes the medical device and or medical device packaging when the sterilizer comes into physical contact therewith. The method is intended for sterilizing unpackaged medical devices and medical devices that are packaged. The present invention is well-suited for sterilizing medical devices with drug and/or polymer coatings that are degradable by steam or irradiation sterilization.

In one embodiment, the present invention includes a method for sterilizing a medical device contained within a sterilization chamber. The method includes placing the medical device in a sterilization chamber configured to maintain set values or ranges of values for temperature, humidity, air pressure, and the like. The temperature inside the chamber is adjusted to be within a range of about 10° C. to about 95° C. The relative humidity inside the chamber is adjusted to be within a range of about 20% to about 90%. The air pressure inside the chamber is adjusted to be within a range of about 10 mbar to about 100 mbar. The temperature, humidity, and air pressure are maintained inside the sterilization chamber for about 0 to about 120 minutes. While maintaining at least the temperature and humidity, a quantity of sterilizer is introduced into the sterilization chamber sufficient to sterilize the medical device in less than about 500 minutes (e.g., about 200 to about 500 minutes). For example, the sterilizer pressure can be from about 200 mbar to about 600 mbar at a concentration from about 100 mg/l to about 1500 mg/l. Finally, the sterilizer is purged from the sterilization chamber.

In one embodiment, the present invention may additionally include a method for using a sterilization system that is configured for sterilizing medical devices and/or medical device packaging through exposure to a sterilizer at predetermined conditions in a substantially closed system such that the sterilizer is capable of sterilizing the medical device and/or medical device packaging. The method includes placing the medical device in a sterilization chamber configured to maintain set values for temperature, humidity, and air pressure. The temperature inside the chamber is adjusted to be within a range of about 45° C. to about 55° C. The relative humidity inside the chamber is adjusted to be within a range of about 45% to about 75%; relative humidity may be adjusted by injecting steam into the sterilization chamber. The temperature and humidity are maintained in the chamber for about 15 minutes to about 60 minutes. The air pressure in the chamber is adjusted to be within the range of about 50 mbar to about 86 mbar. The temperature, humidity, and air pressure are maintained in the chamber for about 15 minutes to about 60 minutes. An amount of a sterilization gas is then introduced into the sterilization chamber such that the pressure within the sterilization chamber is increased to within a range of about 200 mbar to about 600 mbar, wherein the concentration of the sterilization gas is between about 100 mg/l to about 1500 mg/l. The medical device or devices are then incubated at the temperature, humidity, pressure, and sterilization gas concentration for a period of time less than about 330 minutes (e.g., about 270 minutes to about 330 minutes). Finally, the sterilization gas is purged from the sterilization chamber by adjusting the air pressure in the sterilization chamber to be about 50 mbar followed by flushing with an inert gas.

In one embodiment, the present invention may further include a temperature and humidity preconditioning step. The preconditioning step includes placing the medical device in a preconditioning chamber configured to hold set values or a range of values for temperature and relative humidity. The temperature within the preconditioning chamber is adjusted to be in the range of about 10° C. to about 95° C. The relative humidity within the preconditioning chamber is adjusted to be in the range of about 20% to about 90%. The medical device or devices are incubated in the preconditioning chamber for period of time less than about five days (e.g., about 8 hours to about five days). The preconditioning chamber may be a separate chamber or it may be the sterilization chamber.

In one embodiment, the medical device includes a drug-coated and/or polymer-coated stent. Chemical sterilization is well-suited to the sterilization of drug-coated and/or polymer-coated stents because the drug and/or polymer may be degraded in response to heat or irradiation sterilization.

In one embodiment, the medical device is contained within a package. The packaging material is chosen from a group consisting of foils, papers, plastics, plastic coated papers, and combinations thereof. The packaging material can be open or sealed. In instances where the packaging is sealed with a medical device contained within the sealed packaging, the sterilizer sterilizes the packaged medical device by diffusing through the packaging material.

In one embodiment, the sterilizer is a sterilization gas, such as ethylene oxide. When ethylene oxide is used as the sterilizer, the concentration of ethylene oxide in the sterilization chamber is adjusted to be within the range of about 100 mg/l to about 1500 mg/l at a gas pressure of about 200 mbar to about 600 mbar, more preferably from 300 mbar to 400 mbar. Ethylene oxide gas sterilizes medical devices when the gas comes into contact with the device. The sterilizing action of ethylene oxide is accentuated in an environment where temperature, humidity, and pressure are controlled. Given the right temperature, relative humidity, pressure, and ethylene oxide concentration, the gas will effectively sterilize an unpackaged or packaged medical device in less than about 500 minutes (e.g., about 200 minutes to about 500 minutes).

In one embodiment, the sterilization chamber is purged by adjusting the air pressure in the sterilization chamber to be within the range of about 10 mbar to about 100 mbar, and by flushing the sterilization chamber with an inert gas. In the alternative, the sterilization chamber may be purged by opening the sterilization chamber and allowing the sterilizer to diffuse away passively.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates an embodiment of a medical device sterilization system.

FIG. 2 illustrates an embodiment of a medical device sterilization system.

FIG. 3 is a perspective view that illustrates an embodiment of a medical device sheath being held by a clasp.

FIG. 4 is a perspective view that illustrates an embodiment of a clasp having three recesses for releasably retaining a medical device sheath.

FIG. 5 is a perspective view that illustrates an embodiment of a clasp having a holder configured for holding an information sheet.

FIG. 6 is a perspective view that illustrates an embodiment of a clasp having a holder configured for retaining an object in the form of a flushing needle.

FIG. 7 is a perspective view that illustrates an embodiment of a clasp having a holder configured for retaining a flushing needle and a bar for holding two catheter retaining clips.

FIG. 8 is a top view that illustrates an embodiment of a medical device packaging system.

FIGS. 9A-9E are schematic diagrams depicting cross-sectional side views of different embodiments of multi-compartment medical device containers.

FIGS. 10A-10C are top views that illustrate embodiments of multi-compartment medical device containers.

FIG. 11 is a cross-sectional view of an embodiment of a multi-compartment container.

FIG. 12 is a diagram illustrating the incorporation of a medical device and packaging system into a multi-compartment container.

### DETAILED DESCRIPTION OF THE PREFERRED EMBODIMENTS

Generally, the present invention includes methods for sterilizing medical devices and/or medical device packaging. Such methods are intended for sterilizing both packaged and unpackaged medical devices. Packaged and unpackaged medical devices may include devices such as endoscopes, percutaneous translumenal coronary angioplasty (“PTCA”) catheters, drug-coated and/or polymer-coated stents, stent delivery catheters, balloon catheters, and the like. Generally speaking, all medical devices must be thoroughly sterilized prior to use if they are going to be in any way inserted into the body of a patient. The methods described herein can be adapted to sterilize most medical devices. The present invention is well-suited for sterilizing medical devices with delicate components or drug and/or polymer coatings that are degradable by harsh sterilization techniques.

I. The Sterilization System

The present invention can include a sterilization system having a substantially closed sterilization chamber that is configured to maintain and/or adjust values for temperature, humidity, and air pressure. A medical device is sterilized inside the chamber by introducing a quantity of chemical sterilizer at preset values for temperature, humidity, air pressure, and sterilizer concentration. Sterilizer efficacy can be a function of temperature, humidity, air pressure, and sterilizer concentration. Given the appropriate conditions, a quantity of sterilizer effectively sterilizes a medical device in a defined period of time.

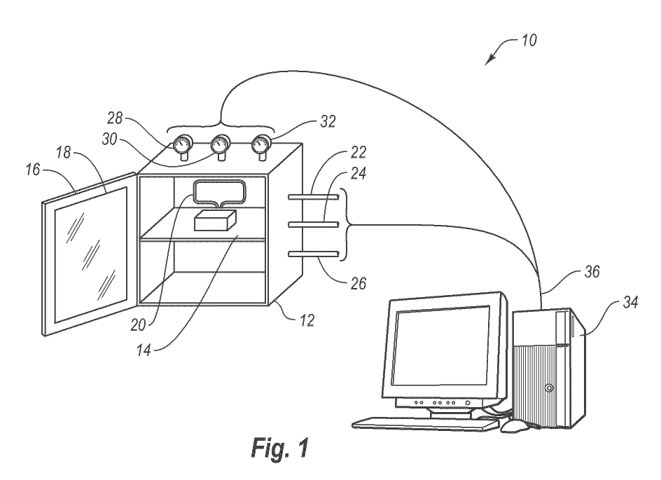

FIG. 1 illustrates a medical device sterilization system 10. The system 10 comprises a sterilization chamber 12 configured to contain a medical device. The chamber 12 contains a sterilization cell 14 configured to contain at least one packaged or unpackaged medical device.

The sterilization chamber 12 can be simple or complex. For example, the sterilization chamber 12 can be a simple polycarbonate or lexan box, or it could be a highly engineered chamber similar to an autoclave chamber. The only requirements for the chamber 12 is that it will hold at least one medical device and that it able to withstand the vacuum, temperature, humidity, and sterilizer used in the method disclosed herein. The chamber 12 includes a door 16 that can be opened or closed. The door 16 is opened to allow insertion of at least one medical device, and the door 16 is closed during the sterilization procedure. The door 16 also includes an airtight seal 18 that allows the chamber to maintain air pressures above or below atmospheric pressure during the sterilization procedure. However, the chamber 12 is generally maintained below atmospheric pressure during the sterilization procedure to prevent sterilizer from escaping from the chamber. That is, if there is a leak, atmospheric air will enter the chamber 12 as opposed to having sterilizer vent out. After the device is inserted, the chamber 12 is a substantially closed and controlled environment. That is, when the chamber 12 is closed, it is configured to maintain system 10 adjusted values for temperature, humidity, air pressure, and sterilizer concentration.

As shown, a number of means for maintaining and/or monitoring the environment inside the sterilization chamber 12 are included. The chamber 12 includes a heating/cooling device 20 that is capable of adjusting the temperature inside the chamber to a range of set values. The chamber 12 is maintained at a relatively constant temperature during the sterilization procedure because the efficacy of the sterilizer is a function of temperature.

The chamber 12 also includes a highly humidified air source 22. An example of highly humidified air is steam. This allows the system 10 to adjust the humidity in the chamber 12 prior to and/or during the sterilization procedure. It is desirable to be able to control the humidity inside the chamber because the sterilizer is able to more effectively penetrate and kill biological organisms in a humidified environment.

The chamber 12 also includes a vacuum source 24. During the procedure, the chamber 12 is initially placed under vacuum to remove excess oxygen from the chamber 12. Because sterilizer efficacy is a function of sterilizer concentration, removal of excess atmospheric air allows the system 10 to carefully control the concentration of sterilizer without having to account for dilution that would otherwise occur because of excess air.

The chamber 12 also includes a sterilizer source 26. According to the method, the sterilizer may be a gas or a liquid. An example of a typical chemical sterilizer is ethylene oxide (EtO). During the sterilization procedure, a sterilizer is introduced into the chamber 12 in a quantity sufficient to sterilize a medical device and/or medical device packaging in a given period of time. The sterilizer source 26 allows the introduction of a known quantity of sterilizer such that the concentration of sterilizer inside the sterilization chamber 12 is known and controlled during the sterilization procedure.

The sterilization chamber 12 can also include various gauges for monitoring the environment inside the chamber 12. A temperature gauge 28 such as a thermometer may be included. This allows the system 10 to monitor the temperature inside the chamber 12. If the temperature is outside the adjusted range, the system 10 can correct the problem by activating the heating/cooling unit 20 inside the chamber 12.

The chamber 12 may also include a humidity gauge 30. This allows the system 10 to correct the relative humidity in the chamber if humidity is outside of the adjusted range. For example, if the relative humidity is too low the system 10 can introduce an appropriate quantity of highly humidified air to raise the relative humidity to the desired level.

The chamber 12 may also include a vacuum gauge 32 to monitor the air pressure inside the chamber 12. This allows the system 10 to correct the air pressure in the chamber 12 if the pressure is outside the adjusted range by either allowing the introduction of air, or sterilizer, or by activating the vacuum source 24.

In addition, the temperature 28, humidity 30, and vacuum 32 gauges may be in communication with a computer 34 that monitors the temperature, humidity, and air pressure inside the chamber 12. As such, the system 10 can include a communication network 36 that links the gauges (28, 30, and 32) to the computer. The network 36 can be electronic, optical, wireless, or combinations thereof. Accordingly, the computer 34 may be programmed to automatically monitor the air pressure, relative humidity, and temperature inside the sterilization chamber 12.

Additionally, the network 36 can communicatively link the computer 34 to the heating/cooling device 20, the highly humidified air source 22, vacuum source 24, and/or the sterilizer source 26. In instances where the computer 34 is programmed to automatically monitor the air pressure, relative humidity, and temperature inside the sterilization chamber 12, the computer 34 can be programmed to control the environment inside the chamber 12. As such, if, for example, the air pressure inside the chamber 12 is outside of adjusted values, the computer 34 can automatically correct the problem by activating the vacuum source 34, allowing the introduction of air, or allowing the introduction of sterilizer. Similarly, the computer 34 can be programmed to control the temperature, humidity, and/or sterilizer concentration inside the chamber 12.

In addition, the computer 34 can be programmed with software to automatically perform the series of steps involved in the sterilization method. As such, the computer 34 can direct the system 10 to automatically perform the steps involved in the sterilization method with minimal user input. Additionally, the computer 34 can be programmed with software that includes sterilization protocols that are specialized for different types of medical devices and/or medical device packaging.

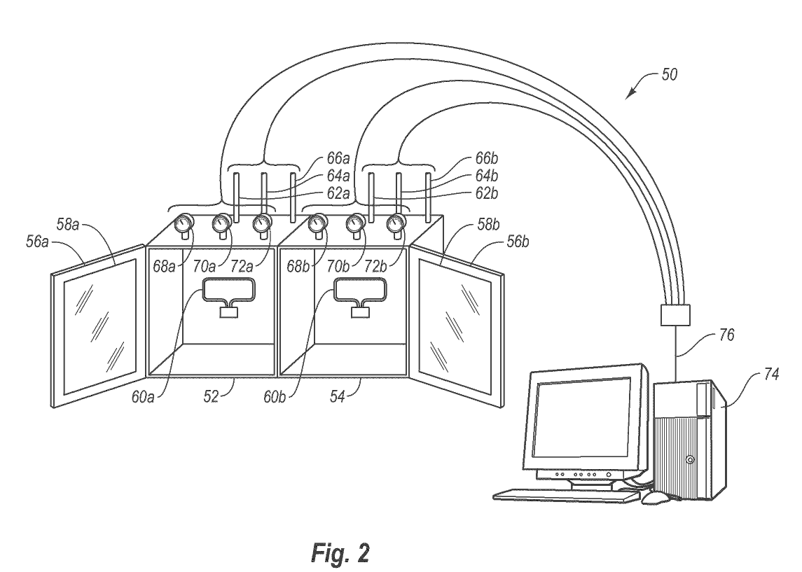

FIG. 2 illustrates an embodiment of a medical device sterilization system 50. In this embodiment, the system 50 consists of a sterilization chamber 52 and a sterilization cell 54 positioned side-by-side. The sterilization chamber 52 and the sterilization cell 54 can be simple or complex. For example, one or the other could be a simple polycarbonate or lexan box or it could be a highly engineered chamber similar to an autoclave chamber. The only requirements for the chamber 52 and the cell 54 is that they are sized to contain at least one medical device and that they are able to withstand the vacuum, temperature, humidity, and sterilizer used in the method disclosed herein. The chamber 52 and the cell 54 each include a door 56a-56b that can be opened or closed. The doors 56a-56b on the chamber 52 and/or the cell 54 are opened to allow insertion of at least one medical device and both doors 56a-56b are closed during the sterilization procedure. Each door 56a-56b also includes an airtight seal 58a-58b that allows the chamber 52 and/or the cell 54 to maintain air pressures above or below atmospheric pressure during the sterilization procedure. However, the chamber 52 and/or the cell 54 are generally maintained below atmospheric pressure during the sterilization procedure to prevent sterilizer from escaping from the chamber 52 or cell 54. That is, if there is a leak, atmospheric air will enter the chamber 52 and/or the cell 54 as opposed to having sterilizer vent out. After the device is inserted, the chamber 52 and/or cell 54 are substantially closed and controlled environments. That is, when the chamber 52 and/or cell 52 are closed, they are configured to maintain system 50 adjusted values for temperature, humidity, air pressure, and sterilizer concentration. Optionally, a door (not shown) can be placed between the chamber 52 and the cell 54 so a medical device can be passed between chamber 52 and cell 54 without opening doors 56a-56b.

As such, a number of means for maintaining and monitoring the environment inside the sterilization chamber 52 and the cell 54 are included. The chamber 52 and cell 54 each include a heating/cooling device 60a-60b that is capable of adjusting the temperature inside the chamber 52 and cell 54 to a range of set values. The chamber 52 and cell 54 are maintained at a relatively constant temperature during the sterilization procedure because the efficacy of the sterilizer is a function of temperature.

The chamber 52 and cell 54 each also include a highly humidified air source 62a-62b. An example of highly humidified air is steam. This allows the system 50 to adjust the humidity in the chamber 52 and/or cell 54 prior to and/or during the sterilization procedure. It is desirable to be able to control the humidity inside the chamber because the sterilizer is able to more effectively penetrate and kill biological organisms in a humidified environment.

The chamber 52 and cell 54 each also include a vacuum source 64a-64b. During the procedure, the chamber 52 and/or the cell 54 are initially placed under a vacuum to remove excess oxygen from the chamber. Because sterilizer efficacy is a function of sterilizer concentration, removal of excess atmospheric air allows the system 50 to carefully control the concentration of sterilizer without having to account for dilution that would otherwise occur because of excess air.

The chamber 52 and cell 54 each also includes a sterilizer source 66a-66b. During the sterilization procedure, a sterilizer is introduced into the chamber 52 and/or the cell 54 in a quantity sufficient to sterilize a medical device and/or medical device packaging in a given period of time. The sterilizer sources 66a-66b allow the introduction of a known quantity of sterilizer such that the concentration of sterilizer inside the sterilization chamber 52 and/or the cell 54 is known and controlled during the sterilization procedure.

The sterilization chamber 52 and the cell 54 can also each include various gauges for monitoring the environment inside the chamber 52 and/or the cell 54. A temperature gauge 68a-68b such as a thermometer may be included. This allows the system 50 to monitor the temperature inside the chamber 52 and/or the cell 54. If the temperature is outside the adjusted range, the system 50 can correct the problem by activating the heating/cooling unit 60a-60b inside the chamber 52 and/or the cell 54.

The chamber 52 and cell 54 may each also include a humidity gauge 70a-70b. This allows the system 50 to correct the relative humidity in the chamber if humidity is outside of the adjusted range. For example, if the relative humidity is too low the system 50 can introduce an appropriate quantity of highly humidified air to raise the relative humidity to the desired level.

The chamber 52 and cell 54 may each also include a vacuum gauge 72a-72b to monitor the air pressure inside the chamber 52 and/or the cell 54. This allows the system 50 to correct the air pressure in the chamber 52 and/or the cell 54 if the pressure is outside the adjusted range by either allowing the introduction of air, sterilizer, or by activating the vacuum source 64a-64b.

In addition, the temperature 68, humidity 70, and vacuum 72a-72b gauges may be in communication with a computer 74 that monitors the temperature, humidity, and air pressure inside the chamber 52 and/or the cell 54. As such, the system 54 can include a communication network 76 that links the gauges (68, 70, and 72) to the computer 74. The network 76 can be electronic, optical, wireless, or combinations thereof. Accordingly, the computer 74 may be programmed to automatically monitor the air pressure, relative humidity, and temperature inside the sterilization chamber 52 and/or the cell 54.

Additionally, the network 76 can communicatively link the computer 74 to the heating/cooling device 60a-60b, the highly humidified air source 62a-62b, vacuum source 64a-64b, and the sterilizer source 66a-66b. In instances where the computer 74 is programmed to automatically monitor the air pressure, relative humidity, and temperature inside the sterilization chamber 52 and/or the cell 54, the computer 74 can be programmed to control the environment inside the chamber 52 and/or the cell 54. As such, if, for example, the air pressure inside the chamber 52 and/or the cell 54 is outside of adjusted values, the computer 74 can automatically correct the problem by activating the vacuum source 74a-74b, allowing the introduction of air, or allowing the introduction of sterilizer. Similarly, the computer 74 can be programmed to control the temperature, humidity, and sterilizer concentration inside the chamber 52 and/or the cell 54.

In addition, the computer 74 can be programmed with software to automatically perform the series of steps involved in the sterilization method. As such, the computer can direct the system 50 to automatically perform the steps involved in the sterilization method with minimal user input. Additionally, the computer 74 can be programmed with software that includes sterilization protocols that are specialized for different types of medical devices and/or medical device packaging.

II. The Sterilization Method

The present invention includes methods for sterilizing medical devices and medical device packaging. The method is intended for sterilizing both packaged and unpackaged medical devices. The present invention is well-suited for sterilizing medical devices with drug and/or polymer coatings that are degraded by steam or radiation sterilization.

In one embodiment, the present invention is a method for sterilizing a medical device contained within a sterilization chamber. The sterilization method is affected by at least four interdependent variables: temperature, humidity, concentration of sterilizer, and time of sterilization. The method includes placing the medical device in a sterilization chamber configured to maintain set values for temperature, humidity, and air pressure. The sterilization chamber may be arranged in one of many possible configurations. However, it is important that the chamber be configured to maintain adjusted values for temperature, relative humidity, and air pressure.

The temperature is generally maintained to be substantially constant throughout the process of sterilization or within a tight range of values. Preferably, the temperature inside the chamber is about 10° C. to about 95° C. More preferably, the temperature range inside the chamber is about 35° C. to about 65° C. Most preferably, the temperature range inside the chamber is about 45° C. to about 55° C. Because the efficacy of the sterilizer is a function of temperature, the method has been designed such that the sterilization will be effective within a given period of time at a given temperature.

Relative humidity is generally maintained to be substantially constant throughout the sterilization procedure, or within a tight range of values. Preferably, the relative humidity inside the chamber is within the range of about 20% to about 90%. More preferably, the relative humidity inside the chamber is within the range of about 35% to about 85%. Most preferably, the relative humidity inside the chamber is within the range of about 45% to about 75%. In a humid environment, the sterilizer is able to more effectively penetrate and kill biological organisms. The method has been designed such that the sterilization procedure will be effective within a given period of time at a given relative humidity.

The air pressure inside the chamber is adjusted to be within a preferred range of about 10 mbar to about 100 mbar. More preferably, the air pressure inside the chamber will be reduced to about 20 mbar to about 80 mbar. Most preferably, the air pressure inside the chamber will be reduced to about 35 mbar to about 55 mbar. This is done for at least three reasons. First, chemical sterilizers can be flammable or explosive when they are combined with oxygen. Second, the method is designed to sterilize medical devices and/or medical device packaging at a given concentration of sterilizer. Removal of excess atmospheric air prevents the dilution of sterilizer by atmospheric air when the sterilizer is introduced into the chamber. Third, sterilizers can be toxic to humans in a manner similar to their toxicity to microscopic organisms. The pressure inside the chamber is generally maintained below atmospheric pressure throughout the procedure so that if there is a leak in the chamber, atmospheric air goes into the chamber as opposed to having toxic gas vent out.

The temperature, humidity, and air pressure are preferably maintained inside the sterilization chamber for about 0 minutes to about 120 minutes. More preferably, the temperature, humidity, and air pressure are maintained inside the sterilization chamber for about 5 minutes to about 75 minutes. Most preferably, the temperature, humidity, and air pressure are maintained inside the sterilization chamber for about 10 minutes to about 60 minutes. The temperature, humidity, and reduced air pressure are maintained for this period of time to allow the contents of the sterilization chamber to achieve temperature and humidity equilibrium and to allow excess oxygen to diffuse out of the medical device or devices contained inside the chamber.

While maintaining the temperature and humidity, a quantity of sterilizer is introduced into the sterilization chamber. As mentioned above, the efficacy of the sterilizer is a function of temperature and relative humidity. The efficacy of the sterilizer is also a function of sterilizer concentration and time. A quantity of sterilizer is introduced into the chamber sufficient to bring the final concentration of sterilizer to a preferred level of about 100 mg/l to about 1500 mg/l at about 200 mbar to about 600 mbar. More preferably, the sterilizer concentration inside the chamber is about 300 mg/l to about 800 mg/l at a gas pressure of about 250 mbar to about 550 mbar. Most preferably, the sterilizer concentration inside the chamber is about 500 mg/l to about 600 mg/l at a gas pressure of about 300 mbar to about 400 mbar. These concentrations are sufficient to sterilize the medical devices and or medical device packaging contained within the chamber within about 200 minutes to about 500 minutes or less. More preferably, the time for sterilization is within the range of about 250 minutes to about 350 minutes. Most preferably, the time for sterilization is within the range of about 270 minutes to about 330 minutes.

After sufficient time has elapsed, the sterilizer is purged from the sterilization chamber using a series of vacuum steps followed by flushing the chamber with an inert gas and/or atmospheric air. This is done so that the sterilizer is properly contained so that there is minimal residue prior to the opening of the chamber and the removal of the medical device and/or medical device packaging. In instances where the sterilizer is not particularly toxic or flammable, the sterilization chamber may be purged by opening the door of the chamber, thus allowing the sterilizer to diffuse away passively.

In one embodiment, the invention may further include a temperature and humidity preconditioning step. A preconditioning step may be desired for any type of medical device. However, preconditioning may, for example, be particularly desirable in cases where the device is large or complex causing an increase in the time required to achieve temperature and humidity equilibrium. The preconditioning step includes placing the medical device in a preconditioning chamber configured to hold set values for temperature and relative humidity. The temperature is adjusted within the preconditioning chamber to be in the range of about 10° C. to about 95° C. More preferably, the temperature is adjusted within the preconditioning chamber to be in the range of about 35° C. to about 65° C. Most preferably, the temperature is adjusted within the preconditioning chamber to be in the range of about 45° C. to about 55° C. The relative humidity within the preconditioning chamber is adjusted to be in the range of about 20% to about 90%. More preferably, the relative humidity within the preconditioning chamber is adjusted to be in the range of about 35% to about 85%. Most preferably, the relative humidity within the preconditioning chamber is adjusted to be in the range of about 45% to about 75%. The medical device or devices are incubated in the preconditioning chamber for about 8 hours to about 5 days or less. More preferably, the medical device or devices are incubated in the preconditioning chamber for about 10 hours to about 3 days. Most preferably, the medical device or devices are incubated in the preconditioning chamber for about 12 hours to about 24 hours. The preconditioning chamber may be a separate chamber or it may be the sterilization chamber.

In one embodiment, the medical device comprises a drug-coated and/or polymer-coated stent. Chemical sterilization is well-suited to the sterilization of drug-coated and/or polymer-coated stents because many drugs and/or polymers may be degraded in response to heat or irradiation sterilization. For example, many drug and/or polymer coatings are degradable and rendered less biologically effective in response to high heat. In addition, high doses of radiation may cause bond scission in many drug or polymer coatings. Bond scission caused by high doses of radiation may lead to significant degradation, radical formation, and possibly toxic byproducts.

In one embodiment, the medical device is contained within a package. The packaging material is chosen from a group consisting of foils, papers, plastics, plastic coated papers, and combinations thereof. In instances where the packaging material is sealed with at least on medical device inside, the sterilizer sterilizes the packaged medical device by diffusing through the packaging material. The sterilizer is able to diffuse through the packaging material because most foils, papers, plastics, plastic coated papers, and combinations thereof are porous enough to allow small molecules like a chemical sterilizer to diffuse through readily. In contrast, the packaging is generally able to maintain the sterile state of the contents after sterilization because, while the packaging material is porous enough to allow small molecule to pass through, large particles like bacteria, spores, or viruses cannot pass through.

In one embodiment, the sterilizer is a sterilization gas. Most commonly, the sterilization gas is ethylene oxide (EtO). EtO is a powerful oxidizing agent. Under conditions of appropriate temperature, humidity, pressure, and sterilizer concentration, EtO is known to kill viruses, bacteria, mold, and fungi, and their spores through contact with gaseous or liquid form. What is more, EtO is highly volatile and no residue of the chemical is left after sterilization. Alternatively, the residual level of EtO can be depleted below regulatory allowable levels (as per ISO 10993-7).

III. Packaging

One skilled in the art would appreciate that many packaging devices and systems can be utilized with the sterilization method disclosed herein. Such packaging devices and systems can be configured to retain elongated medical devices such as catheters, endoscopes, stents and the like. Any of the various packaging devices and systems can be comprised of antistatic materials that inhibit and/or eliminate the formation of static electricity or static charge when the medical device is removed from the packaging device or system. The use of antistatic materials can thereby inhibit sterility from being compromised prior to use. Many examples of packaging devices or systems are described in U.S. patent application Ser. No. 11/353,612, entitled “MEDICAL DEVICE PACKAGING AND ANTISTATIC SYSTEM,” filed on Feb. 14, 2006, which is herein incorporated by this reference.

The packaging devices and systems can be configured to retain elongated medical devices such as catheters, endoscopes, and the like. For example, the packaging device and/or system can include a medical device sheath, medical device clasp, and multi-component container. The medical device sheath can be configured to at least substantially enclose an elongate medical device within an internal lumen to provide protection during storage, transportation, and preparation for use. The medical device clasp can be configured to stably hold the medical device with or without being retained within a sheath in a more compact orientation throughout storage, transportation, and preparation for use. The multi-compartment container can be configured to include a compartment that can store the medical device within the sheath, and a compartment that can store components associated with the medical device.

A. Medical Device Sheath

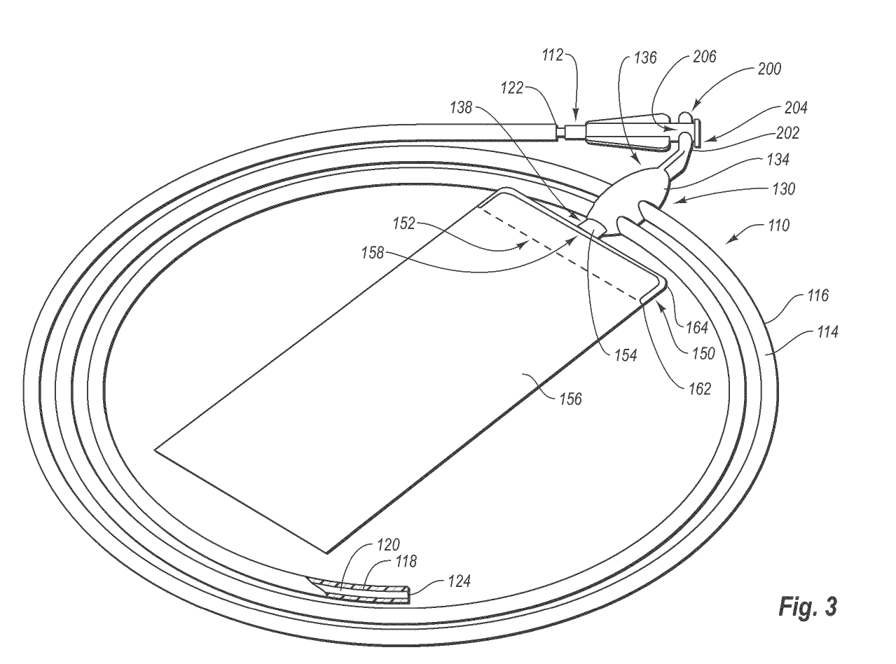

In one embodiment the sterilization method can be used to sterilize a packaged medical device. An example of a packaged medical device is a device contained within a medical device sheath. A medical device sheath can be a protective appliance that is configured to contain a medical device. FIG. 3 provides a perspective view of an embodiment of a medical device sheath 110. As shown, the exemplary embodiment of the medical device is a catheter 112. The medical device sheath 110 can be comprised of an elongate tube 114 having an outer surface 116 and an inner surface 118 defining a lumen 120. The lumen 120 is configured to releasably retain an elongate medical device 112. The sheath 110 can have a first open end 122 and a second open end 124, or at least one of the ends can be sealed or have a sealable cap (not shown). Also, the outer surface 116 and/or inner surface 118 can form a substantially cylindrical shape having near uniform dimensions along the length so as to have a substantially uniform outer diameter and inner diameter. Alternatively, the outer diameter and/or inner diameter can increase or decrease from the first end 122 to second end 124.

The elongate tube 114 can be comprised of at least a first material. Usually, the material is either flexible or resiliently flexible. In the instance the material is flexible, the tube can be positioned into many various orientations and shapes. For example, the material can be a rubbery material that can be bent, wound, coiled, folded, or twisted without deforming the tube or lumen, and retain the orientation without shape memory. Examples of flexible materials include flexible PVC, polyurethane, silicone, liner low-density polyethylene (“LLDPE”), polyethylene, high density polyethylene, (“HDPE”), polyethylene-lined ethylvinyl acetate (“PE-EVA”), polypropylene, latex, thermoplastic rubber, and the like. These materials can be suitable for use as a medical device sheath when configured to have chemical resistance, crack resistance, no toxicity, Food and Drug Administration (“FDA”) compliance, non-electrically conductive, dimensional stability, and/or be sterilized as described herein.

In the instance when the material is flexibly resilient, the material can be similarly bent, wound, coiled, folded, or twisted, but usually tends to return to the original shape, thereby being a material with shape memory. Most of the foregoing materials can be configured to have shape memory by being heat-set with a particular configuration. This can include coiling the tube to have a specified coil diameter and heat-setting the shape. Additionally, nylon, polyurethane, and fluoropolymer (e.g., polytetrafluoroethylene, “PTFE”) materials can be especially suited for being heat-set into a particular shape, such as a coil.

Additionally, there may be instances where it is preferred for the sheath to be prepared from metals, alloys, stainless steel, ceramics, composites, fabrics, and other materials that can be configured in accordance with the foregoing parameters. Also, ribbons prepared from these materials can be used to prepare braid-reinforced sheaths. Moreover, various fabrics prepared from natural materials or synthetic materials (e.g., carbon fibers and aramide fibers) can be used to prepare the reinforcing braid.

In one embodiment, the elongate tube can be additionally comprised of an antistatic material in an amount and distribution so as to inhibit generating static electricity when the elongate medical device is withdrawn from the lumen. The use of antistatic materials will tend to preserve sterility when the device is unsheathed. That is, the device is sterilized in its package, meaning that the packaging and the device are both sterile. Use of an antistatic sheath is intended to prevent the attraction of unsterile particles to the device as it is unsheathed. Antistatic materials are described in more detail below.

B. Medical Device Clasp

In one embodiment, the present invention is intended for the sterilization of packaged medical devices. In the case of elongated medical devices, the device may be packaged in coiled configuration that is designed to save space. The present invention is well-suited to the sterilization of such devices because the sterilizer is capable of sterilizing any surface that it can diffuse around and thereby contact.

An example of a system designed to facilitate the packaging of elongate medical devices is a medical device clasp. A medical device clasp can be an appliance that is configured to hold and/or releasably retain a medical device in a particular conformation. This can be especially useful for elongate medical devices such as those described in connection with the medical device sheath. Additionally, the clip can be configured for use with the sheath so as to hold the sheath in a particular shape or orientation during storage, transportation, and preparation for use.

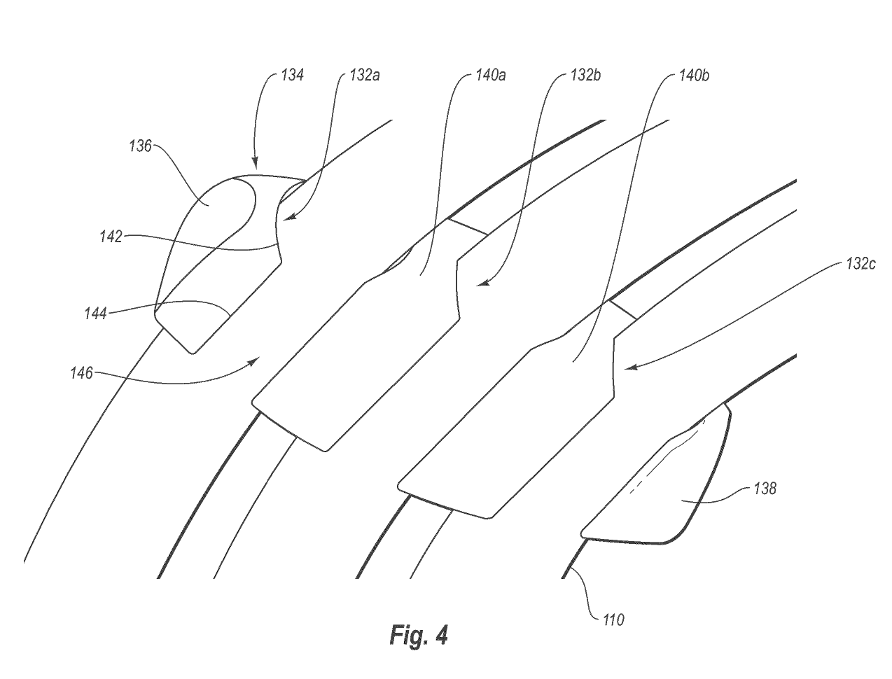

With continued reference to FIG. 3, illustrated is an embodiment of a medical device clasp 130 for releasably retaining a medical device sheath 110. The clasp 130 can be defined by a housing 134 having an outer end 136 and an inner end 138. The housing 134 can be formed to have rounded features that inhibit perforation of packaging enclosing the clasp 130. Additionally, with reference to FIG. 4, the housing 134 can include at least one recess 132a-132c, two recesses (e.g., as shown in FIG. 3), or three recesses 132a-132c as depicted. Optionally, the recesses 132a-132c can be formed into a single side of the housing 134, or on either side of the housing. When including more than one recess 132a-132c, one recess 132c is disposed toward the inner end 138 of the housing 134 with respect to a recess 132a disposed toward the outer end 136 of the housing.

The recesses 132a-132c can be separated by a spacer 140a-b. The outer end 136, inner end 138, and spacers 140a-b can include curved surfaces 142 and lips 144 that cooperate to form the recesses 132a-132c. Each of the recesses 132a-132c can be configured to releasably retain a sheath 110 so that the clasp 130 can hold the sheath in at least a single coil orientation; however, double coil (e.g., as shown in FIG. 3) or triple coil (e.g., as shown in FIG. 4) orientations can also be formed depending on the number of recesses 132a-132c. In the instance of having at least a double coil orientation a single coil passes through each of the recesses 132a-132c, therefore the recesses can be substantially parallel with respect to each other, although different angular orientation of adjacent recesses are possible.

The recesses 132a-132c can have various configurations that allow for receiving and removing the sheath 110. This can include the housing 134 or portions of the housing being comprised of flexibly resilient materials that allow the opening 146 of each recess 132a-132c to be expanded around the sheath 110 and to snap the sheath into place. Alternatively, the housing 134 can be comprised of substantially rigid or resilient materials that allow for the recesses 132a-132c to be slid over the sheath 110 for insertion or removal. In still another configuration, the recess 132a-132c can have flexible, substantially rigid, or rigid/resilient features that aid with selectively retaining the sheath 110. For instance, the recesses 132a-132c can include one or more protrusions that engage with the sheath 110.

Additionally, while the recesses 132a-132c are shown to be substantially circular, other shapes can be employed. In fact, the shape of the recesses 132a-132c can be modified to cooperate with the shape of the medical device and/or sheath being retained therein. This can include the recesses 132a-132c having cross-sectional shapes that range from full circles, ¾ circles, ½ circles, “U” shapes, square shapes, rectangular shapes, and other polygonal shapes.

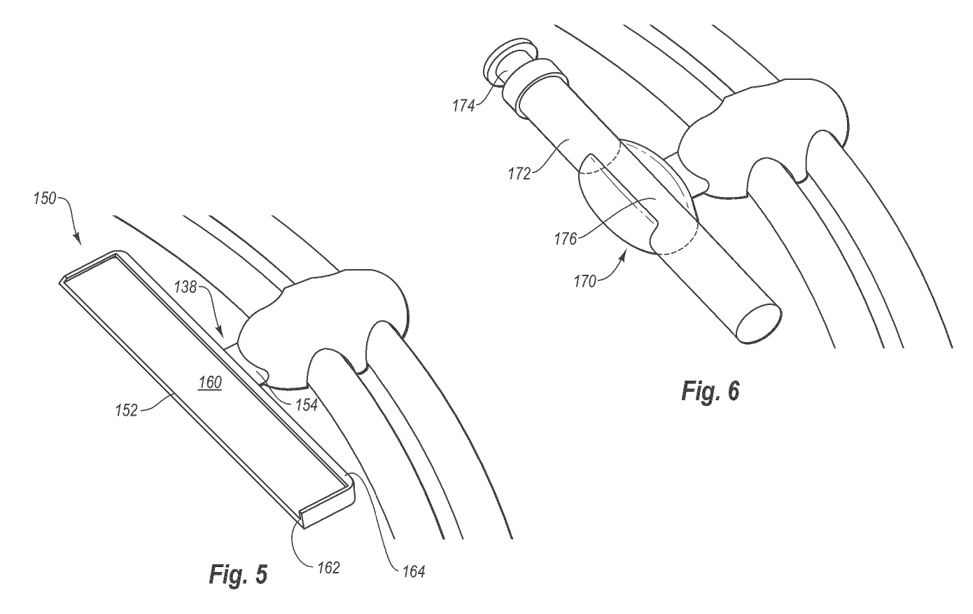

Referring to FIG. 3 and FIG. 5, the clasp 130 can include an inward holder 150 having an inward end 152 and an outward end 154 that is coupled with the inner end 138 of the housing 134. The holder 150 can be configured to releasably retain at least one object 156 in an inwardly generally planar orientation with respect to the coiled elongate tube 114 of the sheath 110. That is, the object 156 can be held and oriented so as to be substantially in a plane with respect to the coiled sheath 110. The clasp 130 is depicted to include a secondary holder 204, which is described in more detail below; however, the clasp can be configured without the secondary holder.

In one embodiment, the inward holder 150 can be configured to releasably retain at least one substantially planar substrate. The planar substrate can be any medium or material (e.g., paper or plastic) that can carry labels, markings, instructions, or other information. For example, catheters are usually supplied with a loose instruction card that is placed within a packaging; however, loose objects can be unfavorable in a medical setting. As such, the holder 150 can hold and retain the planar substrate during storage, transportation, and preparation for use.

The holder 150 can have various configurations for retaining the planar substrate. As illustrated, the holder 150 is substantially in a “T” shape having a center portion 158 with the outward end 154 coupled to the housing 134. The holder 150 can also include a substantially flat base 160 that can provide support to the planer substrate. Additionally, the holder 150 can include an elongated groove 162 extending around a perimeter edge 164. The groove 162 allows the planer substrate to fit therein in a tongue-and-groove configuration. Alternatively, the holder 150 can be configured as a pressure clip, friction clip, crocodile clip, or the like, which are well known in the art to hold planar substrates such as paper.

FIG. 6 is perspective view of another embodiment of an inward holder 170. As depicted, the inward holder 170 can be configured to releasably retain at least one component, such as a generally cylindrical component, that is usable with the elongate medical device. For example, the holder 170 can hold a needle cap 172 containing a flushing needle 174 or other similarly shaped medical devices. Accordingly, the holder 170 can include at least one recess 176, which can be configured similarly as the recesses formed in the housing. Additionally, the holder 170 can be configured as a pressure clip, friction clip, circular friction clip, “U” friction clip, crocodile clip, or the like which are well known in the art for holding and retaining cylindrical objects. Also, the holder 170 can be configured similarly to the recesses 132 of the clasp 130 of FIG. 4.

FIG. 7 is a perspective view illustrating another embodiment of an inward holder 180. As depicted, the holder 180 can include a recess 182 for holding an object 184, and include a bar 186 coupled to the main housing 188 of the holder through a spacer 190. The bar 184 can be configured to have a shape and size that allows for a medical device 192a-192b (e.g., catheter retainer clips) to be coupled therewith. As shown, the catheter clips 192a-192b, which are substantially configured as alligator clips, are clipped to the bar 184. However, the bar 184 may further include a fastener (not shown) for any other medical device associated with the elongate medical device to be retained to the clasp 194. Further, although reference is made to clips being “alligator” type clips, it will be understood by one skilled in the art that various other types of clips or structures capable of selectively attaching to a medical device are possible.

In the illustrated embodiment, the bar 186 and spacer 190 are configured in a “T” conformation that provides a portion of the bar 186 on each side of the intersection with the spacer. This allows for at least one catheter clip 192a to be attached to one side of the bar 186 and at least one catheter clip 192b to be attached to the other side. Additionally, the bar 186 includes an end-cap 196a-192b on each end to aid in holding and retaining the catheter clips 192a-192b during storage, transportation, and preparation for use.

It will be understood by those skilled in the art that various other configurations of the bar 186 are possible. For instance, the bar can include two spaced apart generally cylindrical or curved portions, optionally similar to the bar 186, which are separated by a spacer, such as a spacer similar to spacer 190. The spacer can join or couple the two spaced apart portions along all or a portion of their lengths. In this configuration a groove or recess is formed between the two spaced apart portions. The at least one catheter clip 192a can attach to one of the spaced apart portions, while a portion of at least one catheter clip 192a is received by the groove or recess. End caps can be located on each of the spaced apart portions and/or the spacer to aid in holding and retaining the catheter clips 192a-192b during storage, transportation, and preparation for use. In this configuration, the bar aids in reducing the possibility of rotation movement of at least one catheter clip 192a.

The groove or recess between the two spaced apart portions can be formed as the spacer can have at least one dimension smaller than the diameter of the two spaced apart portions. This can result in the cross-section of the bar having a generally or substantially lemniscate configuration or a cross-section having two generally curved or circular portions and an intermediate portion having a height or thickness smaller than the two curved or circular portions, whether or not that intermediate portion is curved or generally planar in configuration.

Various other configurations of the bar can be provided and identified by one skilled in the art in light of the teaching contained herein to perform the function of preventing or limiting rotational movement of at least one catheter clip. Further, although reference is made herein to cylindrical, curved, or circular, it will be understood that the bar or structure for supporting at least one catheter clip can have various other cross-sectional configurations so long as at least one catheter clip can mount thereto and optionally be received within a recess or groove associated with the bar.

Referring back to FIG. 3, a clasp 130 is described in connection with an embodiment that includes the secondary holder 200. The secondary holder 200 is coupled to the outer end 136 of the clasp 130. The secondary holder 200 is configured to improve the stability of the elongate medical device 112 when held and retained therewith. As shown, the secondary holder 200 includes an arm 202 having an off-axis bend; however, the arm can also be substantially straight. The outer end 204 of the arm 202 can include a recess 206 or is coupled to a member having a recess. The recess 206 can be configured for releasably retaining a portion of the elongate medical device 112 when extending from the first opening 120 of the elongate tube 114. The secondary holder 200 can be any type of retaining device as described herein or well known in the art.

In one embodiment, the secondary holder 200 and recess 206 cooperate to hold a substantially straight portion of the elongate medical device 112. For example, the recess 206 can be shaped and sized to releasably retain a portion of a flushing luer. Additionally, the recess 206 can vary is size and shape to accommodate other types of elongate medical devices for improved stability during storage, transportation, and preparation for use. The recess 206 can be located at various positions of the secondary holder 200. For instance, and as illustrated in FIG. 3, the recess 206 is configured so that a generally upward movement of the flushing luer relative to the recess 206, or generally perpendicular to the plane upon which the medical device sheath 110 rests, decouples the medical device 112 from the secondary holder 200. It will be understood, however, that the recess 206 can also be positioned at a distal end of the secondary holder 200. For instance, the recess 206 can be a generally C-shaped recess at the end of the secondary holder 200 and be configured so that moving the flushing luer in a direction generally outwardly from the secondary holder 200, or generally parallel to the plane upon which the medical device sheath 110 rests, will decouple the flushing luer from the recess 206. It will be understood that the orientation of the recess 206 and the particular direction a user would need to push the flushing luer or medical device 112 to decouple or disengage it from the recess 206 can be varied and would be known to those skilled in the art in light of the teaching contained herein. For instance, other configurations of the secondary holder could have a recess for receiving a portion of the medical device that would entail pushing the medical device at an angular orientation different from generally perpendicular or parallel to the plane upon which the medical device sheath 110 rests.

C. Medical Device Packaging System

The present invention is intended for the sterilization of packaged medical devices. Packaged medical devices may include devices packaged using a medical device packaging system for holding and retaining an elongate medical device during storage, transportation, and preparation for use. Such a packaging system can include multiple clasps having various configurations that cooperate to stably hold and retain the elongate medical device in a coiled orientation. Also, the packaging system can include clasps to hold and retain the various components that function with the elongate medical device. The teaching of the packaging system as it relates to FIGS. 3-7 can also apply to the medical device packaging of FIG. 8.

FIG. 8 is a front view of an exemplary embodiment of a medical device packaging system 220. Such a packaging system 220 can include at least one clasp as generally shown in three different embodiments referenced as 222, 224, and 226. The clasps 222, 224, and 226 can be configured to releasably retain an elongate medical device 228 or sheath 230, as described herein. The packaging system 220 can include only one clasp 226, with or without the secondary holder 204, as generally described in connection with FIG. 3.

In one embodiment, the packaging system 220 can include a plurality of clasps. The number of clasps can be modified in order to achieve the tight-coil orientation of the elongate medical device 228 or sheath 230 depicted in FIG. 8. This can include from two to five or more claps, wherein any embodiment of a clasp depicted and/or described herein can be used. Also, this can include two or more of the embodiments of the clasp 222 as described in connection with FIG. 4. Additionally, various modifications to the clasps described herein can be made and used with the present invention as long as the packaging system 220 can orient the medical device 228 or sheath 230 as depicted and/or described.

In another embodiment, the packaging system 220 depicted and/or described in connection with FIG. 8 can be combined with a multi-compartment container. Briefly, a multi-compartment container can be configured to include a primary compartment to retain the tight-coiled medical device that is held and retained by a plurality of clasps. The multi-compartment container can also include one or more secondary compartments configured to retain components associated with the medical device such as instruments, utensils, information pamphlets, information cards, instruction cards, and the like. An exemplary multi-compartment container is described in more detail below.

D. Multi-Compartment Container

The present invention is well suited for sterilizing medical devices contained in a variety of multi-compartment containers. A multi-compartment container includes a multi-compartment container configured for retaining a medical device in one compartment and at least one other compartment configured to retain components associated with the medical device such as instruments, utensils, information pamphlets, information cards, instruction cards, and the like. At least one of the compartments can be particularly suited to retain a tight-coiled medical device that is held and retained by a plurality of clasps as described herein. The other compartment can have various configurations and orientations depending on the intended contents. The containers can be configured as boxes, contoured envelopes, sleeves, pouches, and the like. As such, the containers can be configured to be rigid, semi-rigid, resiliently flexible, flexible, or the like. At least one of the containers may be comprised of an anti-static packaging material intended to help preserve sterility after the medical device is removed from the package.

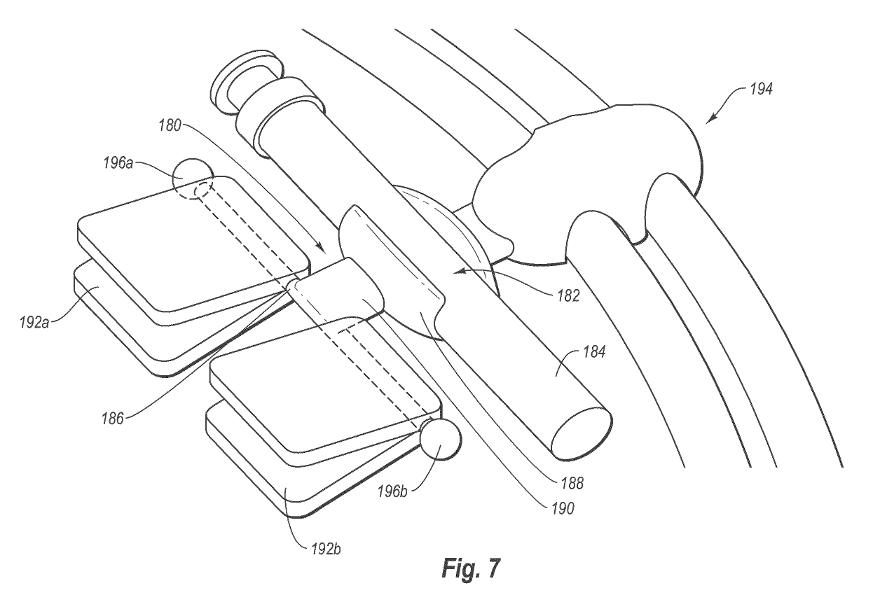

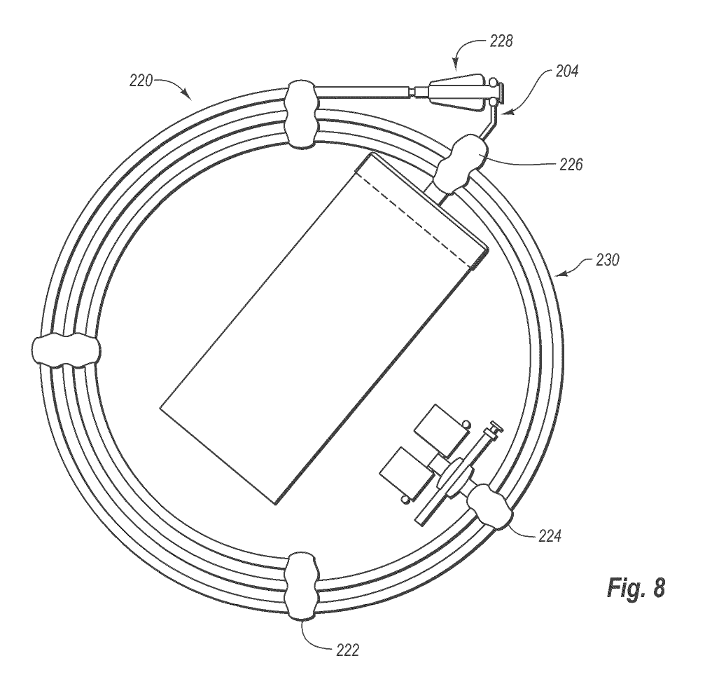

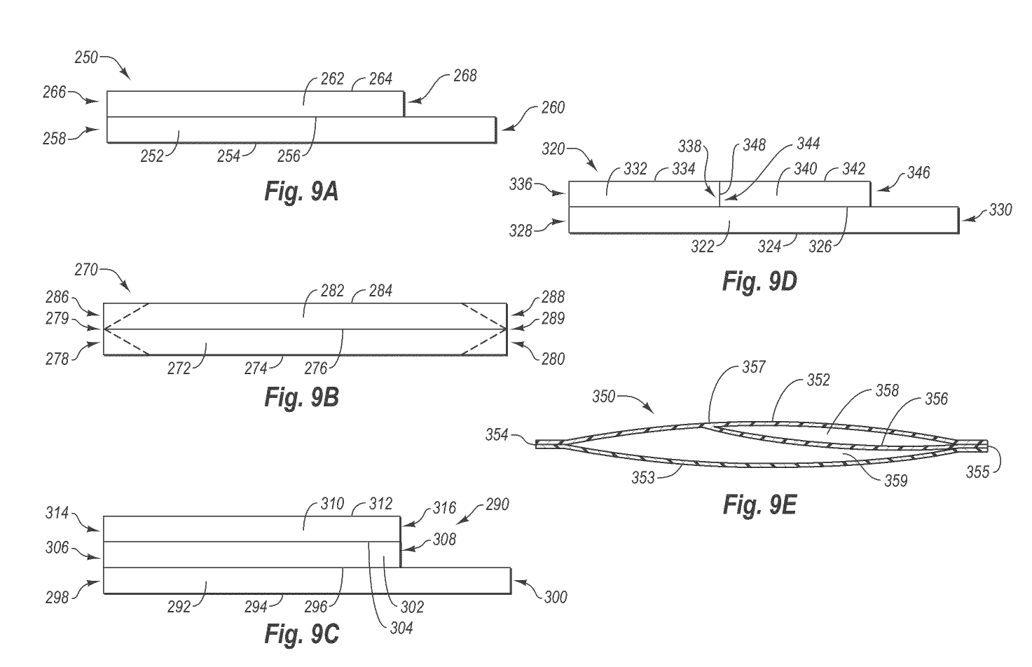

FIG. 9A is a schematic diagram illustrating a cross-sectional side view of an embodiment of a multi-compartment container 250 in accordance with the present invention. As such, the container 250 includes a first compartment 252 defined by a first wall 254, second wall 256, first end 258, and second end 260. Additionally, the container 250 includes a second compartment 262 defined by the second wall 256, third wall 264, first end 266, and second end 268. As such, the first compartment 252 and second compartment 262 are adjacent and share a common wall (e.g., second wall 256). As depicted, the first compartment 252 has a larger volume compared with the second compartment 262. Also, the first compartment 252 and second compartment 262 can share a common peripheral edge (e.g., heat seal, described in more detail below) with the illustrated first end 258 of the first compartment and the first end 266 of the second compartment coming together.

FIG. 9B is a schematic diagram illustrating a cross-sectional side view of another embodiment of a multi-compartment container 270 in accordance with the present invention. As such, the container 270 can include a first compartment 272 defined by a first wall 274, a second wall 276, a first end 278, and a second end 280. Additionally, the container 270 can include a second compartment 282 defined by the second wall 276, a third wall 284, a first end 286, and a second end 288. As such, the first compartment 272 and second compartment 282 are adjacent and share a common wall (e.g., second wall 276) extending from the first ends 278, 286 to the second ends 280, 288. Similar to the embodiment illustrated in FIG. 8A, the first ends 278, 286 can come together in a single first peripheral end 279, and the second ends 280, 288 can come together in a single second peripheral end 289.

Optionally, the multi-compartment container can be in the form of a pouch. Such a pouch can be formed from two flexible walls being joined together at a sealed intersection. The sealed intersection can be formed by heat-sealing the two walls together to form a single edge as a perimeter for each side of both compartments. For example, in FIG. 9B, the first peripheral end 279 can be formed by a seal that joins the first wall 274, second wall 276, and third wall 284 together, as shown by the dashed lines. Similarly, the second peripheral end 289 can be formed by a seal that joins the first wall 274, second wall 276, and third wall 284 together, as shown by the dashed lines. Thus, the first and second ends of the first and second compartments are seals.

FIG. 9C is a schematic diagram illustrating a cross-sectional view of another embodiment of a multi-compartment container 290 in accordance with the present invention. As such, the container 290 can include a first compartment 292 defined by a first wall 294, a second wall 296, a first end 298, and a second end 300. Additionally, the container 290 can include a second compartment 302 defined by the second wall 296, a third wall 304, a first end 306, and a second end 308. As such, the first compartment 292 and second compartment 302 are adjacent and share a common wall (e.g., second wall 296). Also, the container 290 can include a third compartment 310 defined by the third wall 304, a fourth wall 312, a first end 314, and a second end 316. Accordingly, the second compartment 302 and third compartment 310 are adjacent and share a common wall (e.g., third wall 304) extending from the first ends 306, 314 to the second ends 308, 316.

FIG. 9D is a schematic diagram illustrating a cross-sectional view of another embodiment of a multi-compartment container 320 in accordance with the present invention. As such, the container 320 can include a first compartment 322 defined by a first wall 324, a second wall 326, a first end 328, and a second end 330. Additionally, the container 320 can include a second compartment 332 defined by the second wall 326, a third wall 334, a first end 336, and a second end 338. As such, the first compartment 322 and second compartment 332 are adjacent and share a common wall (e.g., second wall 326). Also, the container 320 includes a third compartment 340 defined by the second wall 326, a fourth wall 342, a first end 344, and a second end 346. Accordingly, the first compartment 322 and third compartment 340 are adjacent and share a common wall (e.g., second wall 326). As depicted, the second compartment 332 and third compartment 340 are adjacent and share a common boundary 348 defined by the second end 338 of the second compartment and the first end 344 of the third compartment. The boundary 348 can be a wall, seal, or other similar junction.

FIG. 9E is a schematic diagram illustrating a cross-sectional view of another embodiment of a multi-compartment container 350 comprised of two outer sheets 352, 353, and an inner inserted sheet 356. More specifically, a top outer sheet 352 can be coupled with a bottom outer sheet 353 by having a first end seal 354 and a second end seal 355. Additionally, the corresponding sides (not shown) can also be coupled together with seals that extend from the first end seal 354 to the second end seal 355. An inner insert sheet 356 is disposed between a portion of the top outer sheet 352 and a portion of the bottom outer sheet 353 and sealed thereto at the second end seal 355. The inner insert sheet 356 extends from the first end or the first end seal 354 towards the second end or the second end seal 355 and terminates distal to the first end seal 354. The inner inserted sheet 356 can be coupled with the top outer sheet 352 at the median seal 357. Thus, the top outer sheet 352, bottom outer sheet 353, and inner inserted sheet 356 can form a primary compartment 359, and the top outer sheet 352 and inner inserted sheet 356 can form a secondary compartment 358. The inner insert sheet 356 can also comprise one or more sections of differing materials for different purposes, as will be described in more detail hereinafter. For instance, the inner insert sheet 356 can include at least two different sections of differing materials.

Generally, a multi-compartment container in accordance with the present invention can be prepared from any suitable material for preparing a medical device container. This can include well-known foils (e.g., Mylar®) and different grades of paper, plastics, films, and the like that are used to package medical devices. The external material should be capable of maintaining fluid-tight and/or liquid-tight compartments during storage and transportation of the medical device. Optionally, an insert material can be gas-permeable. Additionally, the materials should be capable of withstanding the sterilization processes described herein without significant loss of its beneficial properties.

In one embodiment, the container can be comprised of a sheet of polymeric material that is formed into a clear film (e.g., transparent), gas-permeable film, soft plastic, and/or hard plastic. The clear material allows for the contents of the container to be visually identified. Gas-permeable films can allow water vapor to be removed from one of the compartments. Additionally, gas-permeable films allow sterilization gas to diffuse through the packaging material where the sterilizer can contact the medical device and thereby sterilize the device. Examples of such films include polyethylene, polyethylene terephthalate, polypropylene, polyesters, cellophane, and the like. Clear materials that can be used for packaging sterile medical devices are well known in the art. Also, the container can be comprised of a translucent or opaque polymeric material, where various polymeric materials are well known in the art to be configured as such.

Additionally, the container can be comprised of a sheet formed from polymeric materials that are metallized by incorporating various metal fibers or particles into the polymeric material. Metallized polymer sheets are also well known for use in medical device packaging.

In one embodiment, the container can be comprised of a sheet formed from fine, continuous, high-density polyethylene fibers. Such fibers are first flash spun, then laid as a web on a moving bed before being bonded together by heat and pressure. Tyvek® (i.e., heat-pressed continuous high density polyethylene fiber) is an example of a protective sheet made of such continuous fibers, and can provide characteristics of paper, plastic, film and/or fabric.

In one embodiment, the container can be comprised of a sheet formed from a foil, such as Mylar®. Mylar® is a biaxially-oriented polyethylene terephthalate (“BOPET”) polyester film that has good tensile strength, chemical resistance, stability, transparency, and electrical insulation. However, other similar foils can also be used.

In one embodiment, the container can be comprised of a plurality of materials. Accordingly, the different walls of a container can each be made of a different material to suit the functionality of an invention. For example, in FIG. 9A, the first wall 254 can be comprised of a foil, the second wall 256 can be comprised of Tyvek®, and the third wall 264 can be comprised of a foil or a clear plastic when it is preferable for the contents to be visible, such as when the contents are cards or pamphlets providing information regarding the medical device.

In another configuration, the first wall 254 and the third wall 264 can be made of one material, while the second wall 256 can be formed by a different material that allows gas and/or liquid to pass between the internal portion or chamber of the first compartment 252 to the second compartment 262, without direct contact between the medical device in the first compartment 252 and the chemical compound and/or component disposed in the second compartment 262. Illustratively, an oxygen and/or liquid scavenger can be placed in the second compartment 262, while a medical device can be placed in the first compartment 252. The second wall 256 can enable the scavenger to perform its desired function (e.g., eliminate oxygen and/or liquid) while preventing direct contact between the scavenger and the medical device.

Additionally, the material forming any of the walls in FIGS. 9A-9E can be comprised of a material suitable for protecting the contents thereof. This can include the Tyvek® being used for a portion or all of the walls in the compartment configured to retain an elongate medical device held in a coiled orientation with the sheath and/or clasps described herein.

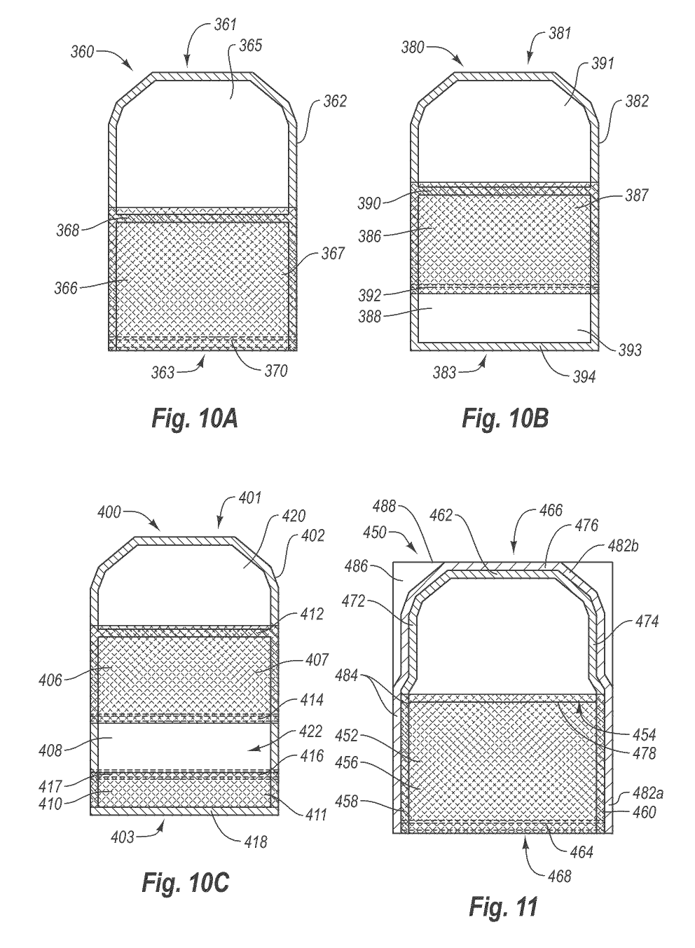

FIG. 10A is a schematic diagram that illustrates a top cut-away view of an embodiment of a multi-compartment container 360. The container 360 can include multiple layers or walls that can be any of a top outer wall (not shown in FIG. 10A for clarity), a bottom outer wall, an inner insert wall, or combination thereof. The container 360 can be in the form of a multi-compartment pouch, which can be referred to as a piggyback container, pouch, or sleeve, where one compartment piggybacks on the other compartment, which can be substantially equal in size or have different sizes, or where one compartment is contained within another compartment, as illustrated in FIG. 9E.

A perimeter seal 362 that extends about the perimeter can define the general shape of the container 360. The container 360 can include a top outer sheet (not shown) and a bottom outer sheet 366 that each extend from the first end 361 to the second end 363. The top outer sheet (not shown) can have a similar configuration to the bottom outer sheet 366. An inner insert sheet 370 is inserted between the top outer sheet and the bottom outer sheet 366. The inner insert sheet 370 is coupled to the top outer sheet at the median seal 368 and portion of the perimeter seal 362 that is between the median seal 368 and the second end 363. Accordingly, the top outer sheet, from the first end 361 to the median seal 368, the inner insert sheet 370 from the median seal 368 to the second end 363, and the bottom outer sheet 366 from the first end 361 to the second end 363, define the primary compartment 365. The top outer sheet, from the median seal 368 to the second end 363, and the inner insert sheet 370 from the median seal 368 to the second end 363 define the secondary compartment 367. The top outer sheet and bottom outer sheet 366 can be comprised of a material such as Mylar® and/or Tyvek®. The inner insert sheet 370 can be a fluid-impermeable material or a fluid and/or gas permeable material such as a polyethylene Tyvek®, or vice versa.

FIG. 10B is a schematic diagram that illustrates a top cut-away view of another embodiment of a multi-compartment container 380. As with container 360, the top outer sheet is not illustrated in FIG. 10B for clarity, but it will be understood by one skilled in the art that the top outer sheet and the bottom outer sheet can have a similar configuration. The container 380 can include a perimeter seal 382 that extends about the perimeter can define the general shape of the container 380. Also, the container 380 can include a top outer sheet and a bottom outer sheet 388 that each extend from the first end 381 to the second end 383. An inner insert sheet 387 can be inserted between the top outer sheet and the bottom outer sheet 388. The inner insert sheet 387 can be coupled to the top outer sheet at the median seal 390 and portion of the perimeter seal 382 that is between the median seal 390 and the second end 383. The inner insert sheet 387 can be comprised of a first material 386 and a second material 394, which are sealed together by an insert seal 392. Accordingly, the top outer sheet from the first end 381 to the median seal 390, the inner insert sheet 387 from the median seal 390 to the second end 383, and the bottom outer sheet 388 from the first end 381 to the second end 383 can define the primary compartment 391. The top outer sheet from the median seal 390 to the second end 383, and the inner insert sheet 387 from the median seal 390 to the second end 383 define the secondary compartment 393. The top outer sheet and bottom outer sheet 388 can be comprised of a material such as Mylar® and/or Tyvek®. The first material 386 of the inner insert sheet 370 can be a fluid-impermeable material, and the second material 394 of the inner insert sheet can be a fluid and/or gas permeable material such as a polyethylene, Tyvek®, or vice versa.

FIG. 10C is a schematic diagram that illustrates a top cut-away view of another embodiment of a multi-compartment container 400. As with containers 360 and 380, the top outer sheet is not illustrated in FIG. 10C for clarity, but it will be understood by one skilled in the art that the top outer sheet and the bottom outer sheet can have a similar configuration. The container 400 can include a perimeter seal 402 that extends about the perimeter can define the general shape of the container 400. Also, container 400 can include a top outer sheet and a bottom outer sheet 418 that each extend from the first end 401 to the second end 403. An inner insert sheet 407 can be inserted between the top outer sheet and the bottom outer sheet 418. The inner insert sheet 407 can be coupled to the top outer sheet at the median seal 412 and portion of the perimeter seal 402 that is between the median seal 412 and the second end 403. The inner insert sheet 407 can be comprised of a first material 406, second material 408, and third material 410. The first material 406 and the second material 408 can be sealed together with a first insert seal 414, and the second material 408 and third material 410 can be sealed together with a second insert seal 416. Accordingly, the top outer sheet from the first end 401 to the median seal 412, the inner insert sheet 407 from the median seal 412 to the second end 403, and the bottom outer sheet 418 from the first end 401 to the second end 403 can define the primary compartment 420. The top outer sheet from the median seal 412 to the second end 403, and the inner insert sheet 407 from the median seal 412 to the second end 403 define the secondary compartment 422. The top outer sheet and bottom outer sheet 418 can be comprised of a material such as Mylar® and/or Tyvek®. The first material 406, second material 408, and third material 410 can be comprised of the same materials, alternating materials or different materials; however, it is preferable for the first material and third material to be fluid impermeable and the second material to be gas and/or liquid permeable.

Alternatively, FIG. 10C can illustrate an embodiment of a container 400 before being utilized as a package. In this configuration, the top outer sheet can extend from the first end 401 to the top opening 417. Additionally, the inner insert sheet 407 can extend from the median seal 412 to the second end 403, and the bottom outer sheet 418 can extend from the first end 401 to the second end 403. After the medical device is inserted into the primary compartment 420 and an associated component, information pamphlet, and/or liquid scavenger is inserted into the secondary compartment 422, the seal at 416 is heat sealed and the extra flap 411 is removed.

An inner insert sheet that includes multiple sections can be prepared to have various configurations. In one embodiment, the individual sections can be coupled together along an end of each section. This results in a single ply sheet having multiple sections. In another embodiment, laying multiple sheets in a sequential order can form the inner insert sheet having multiple sections with some sections having multiple layers.

The foregoing characterizations of an inner insert sheet can be applied to any wall or sheet of a multi-compartment container, such as the top outer sheet, bottom outer sheet, and/or the like. Also, the use of multiple sections having different compositions and/or physical properties can be tailored to the needs of the medical device being retained therein as well as environmental conditions, such as humidity, and/or physical properties. This can include the use of stronger materials at the locations where damage can be more likely to occur such as at the clasps, device ends, and the like. Also, the material for a particular section can be based on the need or desire to visually identify the medical device being retained therein. For example, a wall can have a Tyvek® portion for strength, and a clear polyethylene portion for providing visual identification of the enclosed medical device. Additionally, a fluid permeable section can be used to improve moisture entrapment when a liquid scavenger is placed in the secondary compartment or a compartment other than where the medical device is stored. Cost can also be a factor for using walls with multiple sections having different compositions. This can allow for cheaper materials to be included at locations where there is less susceptibility for puncturing the wall. Thus, the medical device can be placed within the container so that portions that can be more damaging are disposed adjacent to stronger materials.

The container can be prepared in many different shapes and sizes. This can include various shapes and sizes for the primary compartment, and secondary compartment. As such, the container is not limited by shape and size. For example, a container configured for holding a coiled catheter can include an overall length of about 375 mm, an overall width of about 274 mm, and a primary compartment having an inner width of about 231 mm. The secondary compartment can include a length of about 126 mm, an overall width of about 274 mm, and an inner width of about 231 mm.

Additionally, the various seals that impart fluid-tightness to the container and independent compartments can be configured to withstand various forces. This can ensure the individual compartments retain their fluid-tightness during storage and transportation. For example, the width of the seal can range from about 3 mm to about 12 mm, more preferably about 5 mm to about 10 mm, and most preferably about 7 mm. The double seals can be comprised of individual seals having the foregoing dimensions, which can result in a total width that is substantially double the width of a single seal, or wider depending on the space between the first seal and second seal.

FIG. 11 is schematic diagram illustrating a cut-away view of an embodiment of a multi-compartment container 450 in accordance with the present invention. As shown, the multi-compartment container 450 can include a primary compartment 452 having a secondary compartment 454 coupled thereto. A bottom outer sheet 488 can be disposed underneath and coupled with a top outer sheet 486. A head outer seal 476 that extends to a head outer edge seal 482b and a tail outer edge seal 482a can couple the bottom outer sheet 488 to the top outer sheet 486. Optionally, the top outer sheet 486 and bottom outer sheet 488 can be coupled together with a peelable adhesive. An inner insert sheet 456 can be disposed between the top outer sheet 486 and bottom outer sheet 488. The inner insert sheet 456 can be sealed to the top outer sheet 486 at a first insert seal 458 opposite of a second insert seal 460, and at a median seal 478 at the top insert end 454 and a tail end seal 464 at the tail end 468. A double seal 484 can be comprised of outer seals 476, 482a, 482b and the insert seals 458, 460. Optionally, a first inner seal 472 and a second inner seal 474 can also couple the top outer sheet 486 to the bottom outer sheet 488.

The primary compartment 452 can be defined by the top outer sheet 486 from the head end 466 to the median seal 478, the inner insert sheet 456 from the median seal 478 to the tail end 468, and the bottom outer sheet 488 from the head end 466 to the tail end 468. The secondary compartment 454 can be defined by the top outer sheet 486 from the median seal 478 to the tail end 468, and by the inner insert sheet 456 from the median seal 478 to the tail end 468.

In one embodiment, the container 450 can be prepared by sealing the inner insert sheet 456 to the top outer sheet 486 by forming the first insert seal 458 opposite the second insert seal 460, and at the median seal 478. The tail end 468 can be left open. The bottom outer sheet 488 can be placed adjacent to the top outer sheet 486 and the inner insert sheet 456 and sealed with a peelable adhesive that extends around the periphery of the primary compartment 452, which can include the outer seals 476, 482a, 482b and/or the inner seals 462, 472, 474, as well as any of the other seals. Optionally, the outer seals 476, 482a, 482b can be heat seals that form a more secure coupling. After the medical device is placed in the primary compartment 452 and the components, information pamphlets, and/or fluid scavengers are placed in the secondary compartment 452, the tail end seal 464 can be formed by a heat seal, peelable seal, combination thereof, or the like.

In one embodiment, the outer seals can be formed by an external material that covers substantially the entire container so as to form an external surface. As such, the double seals can be formed of the external material as well as the bottom outer sheet, top outer sheet, and/or inner insert sheet. The external material can be any suitable material for forming and external covering on medical devices, electronics, and the like; however, substantially any material can be used as the external surface in instances where the primary compartment and secondary compartment are sealed from the external material. For example, the external material can be any material described herein, where preferable materials include foils, Mylar®, Tyvek®, cellophanes, hard plastics, soft plastics, and like covering materials.

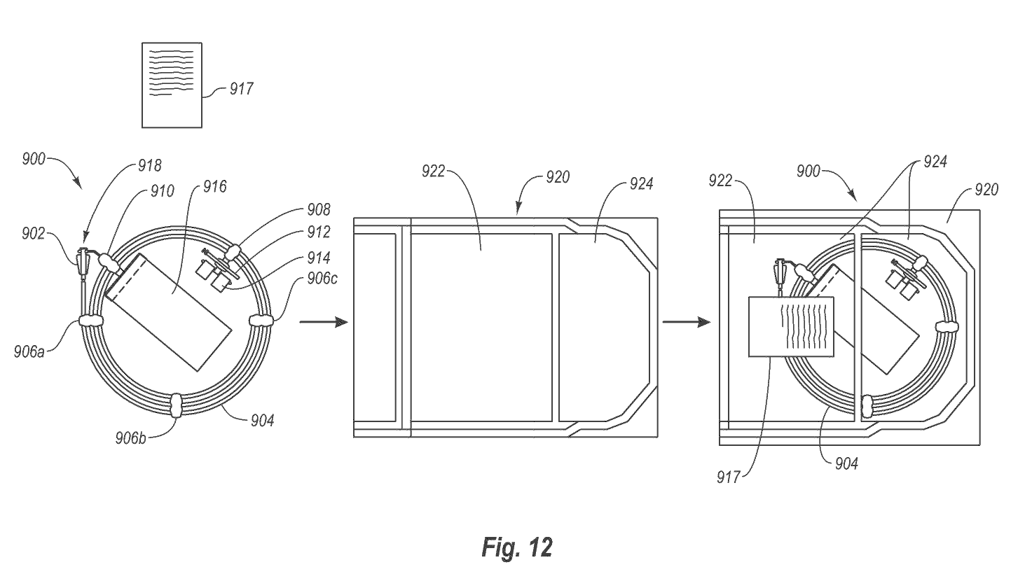

FIG. 12 is a schematic diagram of an embodiment of a packaging system 900. As depicted, the system 900 can include an elongate medical device 902 (e.g., catheter) encased within a sheath 904, which is held in a coiled orientation with clasps 906a-906c, 908, 910. Clasp 908 can be configured to hold a flushing needle 912 and catheter clips 914, and clasp 910 can be configured to hold a substrate 916 and a protruding end 918 of the medical device 902. The medical device 902 can be packaged in a container 920 with an additional component, information pamphlet, and/or liquid scavengers generally designated by the pamphlet 917. The container 920 can be comprised of at least a first compartment 924 configured to hold and retain the coiled medical device 902 in the sheath 904, and an optional second compartment 922 can be used for holding the additional components, information pamphlets 917, and/or liquid scavengers. Optionally, the container can include only a single compartment or any number of compartments as needed.

It has been discovered that the amount of particulate matter capable of attaching to a medical device can be reduced through the selection of antistatic materials that reduce the formation of a static electric charge on the medical device. Additionally, a multi-compartment container can be comprised of an antistatic material. Accordingly, the multi-compartment container containing the medical device within the sheath can be sterilized as a single component, wherein the container and protects the medical device and sheath from contamination during storage and shipping without compromising sterility.

E. Antistatic Components

One embodiment of the present invention is a method for sterilizing packaged medical devices. However, there are particles in the air such as dust or spores that would compromise the sterility of the device if they were attracted to the device after it is removed from the packaging. As such, the present invention is facilitated if antistatic materials are to be included in the medical packaging, containers, and systems described herein. Additionally, the antistatic materials can also be included in a medical device (e.g., catheter, endoscope, and the like) to inhibit the generation of static electricity. The use of antistatic materials in the medical devices and/or packaging can inhibit the generation of static electricity when the medical device is withdrawn from container and sheath. While the antistatic materials are generally described in connection to the sheath and containers of a packaging system, it should be recognized they can also be utilized in any medical device for enhanced antistatic characteristics.

In one embodiment, a sheath in accordance with the present invention can be comprised of a first material, such as a plastic, metal, ceramic, combination thereof or material described herein in order to provide a flexible and strong protective package. Accordingly, the sheath can have the properties as described herein. Additionally, the sheath can also include an antistatic material distributed throughout the first material so as to prove an antistatic medical device packaging. The sheath can be configured to include the first material and the second material in a manner that does not alter the properties of the medical device disposed within the sheath by using materials that are substantially inert with respect to the material of the medical device. Also, the materials that comprise the sheath can result in a lumen that does not significantly interact with lubricants commonly applied to medical devices, which can be a consequence of using the chemically stable materials described herein.

In one embodiment, the first material can be any of the materials described herein in connection with the sheath so as to provide the desired features of the sheath. For example, the first component can be preferably polyethylene or high density polyethylene; however, other materials can be used.

The antistatic material can by any type of material that inhibits the formation of static electricity by being electrically non-conductive, or being an electron scavenger. For example, the antistatic material can be selected from the group consisting of polytetrafluoroethylene (“PTFE”), fluorinated ethylene-propylene polymer (“FEP”), carbon-filled polymer, glycerolmonostearate, ethoxylated alkylamine, nonionic ethoxylated alkylamine, lauric diethanol amine, alkyl sulfonates, alkyl dimethyl benzyl ammonium chloride/bromide, anionic aliphatic sulfonate/phosphates, quaternary ammonium compounds, glass-impregnated polystyrene, glass-impregnated acrylonitrile butadiene styrene polymers, antistatic polycarbonate, cationic scavengers, and combinations thereof. Additionally, in some instances it can be preferred for the antistatic material to be selected from the group consisting of polytetrafluoroethylene, fluorinated ethylene-propylene polymer, carbon-filled polymer, glycerolmonostearate, ethoxylated alkylamine, and combinations thereof.

The antistatic material can be combined into the sheath, clasps, container, or other packaging material for the medical device so as to inhibit the generation of static electricity. This can include forming the sheath, clasps, container, or other packaging material to include the antistatic material at from about 0.5% to about 49% by weight, more preferably from about 5% to about 39% by weight, and most preferably from about 10% to about 29% by weight.

In another embodiment, a hygroscopic material can be combined with the first material. Optionally, the hygroscopic material can be present in an amount and distribution so as to absorb water present within the sheath and/or container. The presence of a hygroscopic material can serve to bind excess water, and can also be a supplemental antistatic agent. In part, this is because water bound within the hygroscopic material may be inhibited from contributing to the generation of static electricity and/or can keep the conductivity relatively constant. Also, the use of a hygroscopic material can allow for the contents of the sheath and/or container to be maintained reasonably independent of the humidity at which the package is stored. Moreover, the hygroscopic material can inhibit degradation over time and/or in response to environmental conditions.

The hygroscopic material can be any material that binds and retains water. In some instances it can be preferred for the hygroscopic material to be a hydroscopic scavenger. For example, the hygroscopic scavenger can be selected from the group consisting of phosphorous pentoxide, ethanol, methanol, glycerin, sodium hydroxide, H₂SO₄, ZnSO₄, CaCl₂, SiO₂, NaNO₃, CaSO₄, and combinations thereof.

The hygroscopic material can be combined into the sheath, clasps, container, or other packaging material for the medical device for any of the purposes described herein. This can include forming the sheath, clasps, container or other packaging material to include the hygroscopic material at from about 0.05% to about 10% by weight, more preferably from about 0.5% to about 5% by weight, and most preferably from about 1% to about 2.5% by weight.

Additionally, an embodiment of the present invention can include a method of reducing static electricity in a medical device. Such a method can include selecting an appropriate material such as a major component of a sheath and/or container. This can include selecting the major component material from polyethylene, high density polyethylene, or the like as described herein. In any event, the major component material can be a material that does not contribute to the generation of static electricity. The method can then include selecting an antistatic material to be a minor component of the sheath and/or container. This can also include selecting an effective amount of antistatic material so as to inhibit the generation of static electricity in the medical device during storage, transportation, and preparation for use. The sheath and/or container can be formed from at least the major component material and minor component antistatic material. Also, the medical device, which can optionally also include an antistatic material, can then be packaged in the sheath and/or container and sterilized.

In another embodiment, the present invention can include a method of reducing particulate matter on a medical device. Such a method can include forming a medical device, sheath, and/or container to include an effective amount of an antistatic material distributed within a major component material that does not contribute to static generation. The method also includes packaging the medical device in the sheath, and inserting the sheath in the container. The medical device and sheath can be sterilized at any time, which can include before or after being inserted within the container. Thus, withdrawal of the medical device from the sheath and/or container can have reduced static electricity generation.

Additionally, it should be recognized that although the present invention has been generally described in connection with an elongate medical device, such as a endoscope or catheter, it is contemplated that the sheath and/or container could be configured for and utilized with other types of medical devices where there is a desire to reduce the formation of static electricity. For example, packing trays for medical devices may be prepared from the majority component materials and/or antistatic materials as described herein. This can serve to reduce the formation of static electricity formation when any type of medical device is removed from its packaging.

These and other embodiments and features of the present invention will become more fully apparent from the following examples and appended claims, or may be learned by the practice of the invention as set forth hereinafter.

### EXAMPLES

### Example 1

The following presents one example of how the sterilization method may be practiced. The present invention may include a sterilization system configured for sterilizing medical devices and/or medical device packaging through exposure to a sterilizer at predetermined conditions in a substantially closed system such that the sterilizer is capable of sterilizing unsterile portions of the medical device and/or medical device packaging. The method comprises placing the medical device in a sterilization chamber configured to maintain set values for temperature, humidity, and air pressure.

Medical devices and/or medical device packaging are sterilized by a chemical sterilizer in a process that includes placing the unpackaged and/or packaged medical devices and/or medical device packaging into a sterilization chamber. The inside of the sterilization chamber is a controlled environment where the sterilization takes place under controlled conditions.

After the devices and/or packaging are inserted, the temperature inside the chamber is preferably adjusted to be within a range of about 45° C. to about 55° C. Most preferably, the temperature inside the chamber is about 50° C. The relative humidity inside the chamber is preferably adjusted to be within a range of about 45% to about 75%. Most preferably, the relative humidity inside the chamber is about 60%. The relative humidity inside the chamber is generally adjusted by injecting small quantities of steam. The temperature and humidity are maintained in the chamber for about 15 minutes to about 60 minutes to allow the medical device or devices contained therein to achieve temperature and humidity equilibrium with the rest of the chamber. Some small devices may equilibrate more quickly, whereas larger or more elaborately packaged devices may take longer to equilibrate.

After this initial temperature and humidity equilibration step, the air pressure in the chamber is preferably adjusted to be within the range of about 50 mbar to about 85 mbar. Most preferably, the pressure inside the chamber is adjusted to about 65 mbar. Insofar as possible, the temperature and humidity should be maintained during this vacuum step. The temperature, humidity, and air pressure should be maintained in the chamber for about 15 minutes to about 60 minutes.

An amount of a sterilization gas is then introduced into the sterilization chamber. Preferably, a quantity of EtO is introduced into the chamber such that the pressure within the sterilization chamber is increased to about 320 mbar to about 360 mbar, and the concentration of the sterilization gas is between about 500 mg/l to about 600 mg/l. Most preferably, the EtO injection increases the pressure in the chamber to about 340 mbar at an EtO concentration of about 550 mg/l. Preferably, the medical device or devices are incubated at the temperature, humidity, pressure, and sterilization gas concentration for a period of time less than about 330 minutes. Most preferably, the incubation/sterilization time is about 300 minutes.

Finally, the sterilization gas is purged from the sterilization chamber by reducing the air pressure in the sterilization chamber to about 50 mbar followed by flushing with an inert gas. This purging step is repeated at least twice.

### Example 2

The following table presents an example for a full sterilization cycle that includes a preconditioning step.

TABLE 1
Summary of an Embodiment of a Full Sterilization Cycle

| Stage | Phase | Parameter | Set Point | Tolerance |
|---|---|---|---|---|
| Precon | Outer preconditioning | Temperature | 30° C. | ±5° C. |
| | area | Time | 12 hours | 12 hours |
| Precon | Preconditioning cell | Temperature | 50° C. | ±5° C. |
| | | Relative humidity | 60% | ±15% |
| | | Dwell time | 12 hours | 0-12 hours |
| | | Transfer time | 1 hour | Maximum |
| Chamber | Initial vacuum | Evacuate to | 68 mbar | ±18 mbar |
| | | Evacuation time | 15-60 min. | |
| | Leak test (vacuum hold) | Pressure change | 0 mbar | ±18 mbar |
| | Steam injection | Pressure change | 36 mbar | ±18 mbar |
| | | Time | 0-5 min. | |
| | Nitrogen injection | Pressure | 816 mbar | ±18 mbar |
| | | Time | 15-45 min. | |
| | 2nd vacuum | Pressure | 68 mbar | ±18 mbar |
| | | Time | 15-60 min. | |
| | Steam injection | Pressure change | 36 mbar | ±18 mbar |
| | | Time | 0-5 min. | |
| | | Dwell time | 15 min | Minimum |
| | Gas injection (EtO) | Pressure change | *340 mbar | ±18 mbar |
| | | Time | 15-60 min | |
| | | Gas temp. | 30-60° C. | |
| | EtO gas exposure | Chamber temp. | 50° C. | ±3° C. |
| | | Dwell time | 300 min. | 30 min. |
| | | Gas weight | 35-43 kg | |
| | 1st post exposure vacuum | Pressure | 68 mbar | |
| | | Time | 15-60 min. | |
| | Nitrogen wash 1 | Pressure | 816 mbar | ±18 mbar |
| | | Time | 15-45 min. | |
| | 2nd post exposure | Pressure | 68 mbar | ±18 mbar |
| | vacuum | Time | 15-60 min. | |
| | Air wash 1 | Pressure | 816 mbar | ±18 mbar |
| | | Time | 15-45 min. | |
| | 3rd post exposure | Pressure | 68 mbar | ±18 mbar |
| | vacuum | Time | 15-60 min. | |
| | Air wash 2 | Pressure | 816 mbar | ±18 mbar |
| | | Time | 15-45 min. | |
| | 4th post exposure | Pressure | 68 mbar | ±18 mbar |
| | vacuum | Time | 15-60 min. | |
| | Air wash 3 | Pressure | 816 mbar | ±18 mbar |
| | | Time | 15-45 min. | |
| | 5th post exposure | Pressure | 68 mbar | ±18 mbar |
| | vacuum | Time | 15-60 min. | |
| | Air wash 4 | Pressure | 1 bar | ±18 mbar |
| | | Time | 15-45 min. | |
| | Chamber to cell | Transfer time | 1 hour | Maximum |
| Degassing | Primary cell | Temperature | 50° C. | ±5° C. |
| | | Time | 8 hours | Minimum |

*The concentration of EtO during sterilization is 557 mg/l at a pressure of 340 mbar at 50° C.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of claims are to be embraced within their scope.

What is claimed is:

1. A method for sterilizing a medical device within a sterilization chamber, comprising:
placing the medical device in the sterilization chamber configured to maintain set values for temperature, humidity, and air pressure;
adjusting a temperature inside the sterilization chamber to be within a range of about 10° C. to about 95° C.;
adjusting a relative humidity inside the sterilization chamber to be within a range of about 20% to about 90%;
introducing an amount of a sterilizer into the sterilization chamber sufficient to sterilize the medical device an to achieve a pressure; and
purging the sterilizer from the sterilization chamber.

2. The method according to claim 1, further comprising at least one of the following:
adjusting an air pressure in the sterilization chamber to be within a range of about 10 mbar to about 100 mbar before the sterilizer is introduced; or
increasing the pressure in the sterilization chamber with the sterilizer to be within a range of about 200 mbar to about 600 mbar.

3. The method according to claim 1, further comprising substantially maintaining at least one of the temperature, humidity, or pressure in the sterilization chamber.

4. The method according to claim 3, wherein the maintaining is for about 10 to about 120 minutes.

5. The method according to claim 1, further comprising sterilizing the medical device with the sterilizer for about 200 to about 500 minutes.

6. The method according to claim 1, wherein the medical device is contained within a package.

7. The method according to claim 6, further comprising sterilizing the packaged medical device by diffusion through a packaging material.

8. The method according to claim 1, wherein the sterilizer is ethylene oxide.

9. The method according to claim 7, further comprising adjusting a concentration of ethylene oxide in the sterilization chamber to be within a range of about 100 mg/l to about 1500 mg/l at a gas pressure of about 300 mbar to about 500 mbar.

10. The method according to claim 1, wherein the medical device comprises a drug and/or polymer-coated stent, and the drug and/or polymer is degraded in response to heat or radiation sterilization.

11. The method according to claim 1, further comprising injecting steam into the sterilization chamber to adjust the humidity.

12. The method according to claim 1, further comprising a preconditioning step, the preconditioning step comprising:
placing the medical device in a preconditioning chamber configured to hold set values for temperature and relative humidity;
adjusting the temperature within the preconditioning chamber to be in a range of about 10° C. to about 95° C.;
adjusting the relative humidity within the preconditioning chamber to be in a range of about 20% to about 90%; and
incubating the medical device in the preconditioning chamber.

13. The method according to claim 12, wherein the preconditioning chamber is the sterilization chamber.

14. The method according to claim 1, wherein the purging comprises at least one of the following:
adjusting an air pressure in the sterilization chamber to be within a range of about 10 mbar to about 100 mbar; and
flushing the sterilization chamber with an inert gas.

15. In a sterilization system configured for sterilizing medical devices and/or medical device packaging through exposure to a sterilizer at predetermined conditions in a substantially closed system such that the sterilizer is capable of sterilizing unsterile portions of the medical device and/or medical device packaging, a method for sterilizing the medical device and/or medical device packaging with the sterilizer such that the sterile medical device and/or medical device packaging is usable in a medical procedure, the method comprising:
placing the medical device in a sterilization chamber;
adjusting a temperature inside the sterilization chamber to be less than about 100° C.;
adjusting a relative humidity inside the sterilization chamber to be within a range of about 20% to about 90%;
adjusting an air pressure in the sterilization chamber to be within the range of about 10 mbar to about 100 mbar;
introducing an amount of a sterilization gas into the sterilization chamber such that the concentration of the sterilization gas is between about 100 mg/l to about 1500 mg/l at a pressure;
incubating the medical device at substantially the temperature, humidity, pressure, and sterilization gas concentration so as to sterilize the medical device; and
purging the sterilization gas from the sterilization chamber.

16. The method according to claim 15, further comprising maintaining at least one of the temperature, relative humidity, and air pressure inside the sterilization chamber for about 15 to about 60 minutes.

17. The method according to claim 15, further comprising maintaining the sterilization gas at a pressure of about 200 mbar to about 600 mbar.

18. The method according to claim 17, wherein the sterilization gas pressure is about 300 mbar to about 400 mbar.

19. The method according to claim 15, wherein the medical device is contained within the medical device packaging.

20. The method according to claim 15, wherein the sterilization gas is ethylene oxide.

21. The method according to claim 15, wherein the medical device comprises a drug and/or polymer-coated stent, and the drug and/or polymer is degraded in response to heat or radiation sterilization.

22. The method according to claim 15, further comprising a preconditioning step, the preconditioning step comprising:
placing the medical device in a preconditioning chamber configured to hold set values for temperature and relative humidity;
adjusting the temperature within the preconditioning chamber to be in a range of about 10° C. to about 95° C.;
adjusting the relative humidity within the preconditioning chamber to be in a range of about 20% to about 90%; and
incubating the medical device in the preconditioning chamber.

23. The preconditioning step according to claim 20, wherein the preconditioning chamber is the sterilization chamber.

24. The method according to claim 15, further comprising adjusting the relative humidity by injecting steam into the sterilization chamber.
